(19) United States
(12) Patent Application Publication
(10) Pub. No.: US 2024/0366666 A1
(43) Pub. Date: Nov. 7, 2024

(54) METHODS AND COMPOSITIONS FOR ENHANCING THE CYTOTOXICITY OF GAMMA/DELTA-T CELLS

(71) Applicant: PhosphoGam, LLC, Durham, NC (US)

(72) Inventor: Richard D. Lopez, Durham, NC (US)

(73) Assignee: PhosphoGam, LLC, Durham, NC (US)

(21) Appl. No.: 18/553,947

(22) PCT Filed: Mar. 8, 2022
(86) PCT No.: PCT/IB2022/052065

§ 371 Date: Oct. 4, 2023

Related U.S. Application Data
(60) Provisional application No. 63/172,257, filed on Apr. 8, 2021.

(51) Int. Cl. A61K 35/17; A61K 39/00; A61K 45/06; C07K 14/725; C07K 16/28; C12N 5/0783

(52) U.S. Cl. CPC A61K 35/17; A61K 39/4611; A61K 39/4631; A61K 39/4644; A61K 45/06; C07K 14/7051; C07K 16/28; C12N 5/0637; C07K 2317/31; C07K 2317/75; C12N 2510/00

(57) ABSTRACT

Compositions and methods for enhancing the cytotoxicity of gamma/delta-T cells (γδ-T cells) by engineering or modifying through non-genetic means the γδ-T cells to express ligands and/or agonist antibodies or fragments thereof which recognize and bind or engage death receptors on tumor cells are provided herein. In this manner, γδ-T cells can be modified to enhance cancer therapy. When the engineered or non-genetically modified γδ-T cells engage or bind their target(s), they will now be more effective at destroying the cancer cells. Compositions and methods for treating cancer in a subject are therefore provided herein. Such methods comprise the administration of engineered or non-genetically modified γδ-T cells that are expressing a death receptor ligand or an agonist death receptor antibody or fragment thereof to a subject.

### FIELD OF THE INVENTION

This invention relates to the field of immunology and methods of treating cancer.

### BACKGROUND OF THE INVENTION

Unlike classical (conventional) αβ-T cells that recognize specific peptide antigens presented by major histocompatibility complex (MHC) molecules, γδ-T cells (gamma/delta-T cells) in contrast appear to recognize generic determinants expressed by cells that have become dysregulated as a result of either malignant transformation or viral infection (Kabelitz D. et al. Cancer Res. 2007; 67 (1): 5-8; Silva-Santos B, et al., Eur J Immunol. 2012; 42 (12): 3101-5; Vantourout P and Hayday A. Nature reviews Immunology 2013; 13 (2): 88-100). γδ-T cells have the innate ability to recognize and kill a broad spectrum of tumor cell types, in a manner that does not require the existence of bona fide tumor-specific antigens. As such, there is a need to develop therapeutic strategies to utilize γδ-T cells in the treatment of cancer.

### SUMMARY OF THE INVENTION

Compositions and methods for enhancing the cytotoxicity of gamma/delta-T cells (γδ-T cells) are provided. The compositions comprise modified γδ-T cells that activate cell death pathways in tumor cells. In one embodiment the γδ-T cells are engineered or genetically modified to express ligands and/or agonist antibodies or fragments thereof which recognize and bind or engage death receptors on tumor cells are provided. Additionally, the γδ-T cells may be modified by attaching moieties that activate cell death pathways. In this manner, γδ-T cells can be modified to enhance cancer therapy. γδ-T cells naturally recognize cancer cells and destroy them. In some embodiments, the modified γδ-T cells comprise a death receptor ligand and/or agonist antibodies or fragments thereof on the surface of the γδ-T cells. When the modified γδ-T cells engage or bind their target(s), they will now be more effective at destroying the cancer cells. In some embodiments, tumor targeting antibodies or moieties promoting tumor cell death can be attached to the γδ-T cells by chemical means. Compositions and methods for treating cancer in a subject are provided. Such methods comprise the administration of modified γδ-T cells that comprise a death receptor ligand or an agonist death receptor antibody or fragment thereof to a subject.

### DETAILED DESCRIPTION OF THE INVENTION

Before describing the present invention in detail, it is to be understood that this invention is not limited to particular embodiments, which can, of course, vary. It is also to be understood that the terminology used herein is for the purpose of describing particular embodiments only, and is not intended to be limiting.

Many modifications and other embodiments of the inventions set forth herein will come to mind to one skilled in the art to which these inventions pertain having the benefit of the teachings presented in the foregoing descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

As used herein, the singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description.

### I. Overview

Herein provided are compositions and methods for enhancing the cytotoxicity of gamma/delta-T cells (γδ-T cells). Compositions comprise modified γδ-T cells that are programmed to target tumor cells. By “modified γδ-T cells” is intended γδ-T cells that comprise ligands and/or agonist antibodies or fragments thereof that recognize and bind or engage death receptors on tumor cells. The modified γδ-T cells are created by genetic modification or by chemical means. Death receptors are important in inducing apoptosis and are activated according to the methods of the invention as potential therapeutic agents. See, Kumar et al. (2005) International J of Surgery 3 (4): 268-277, herein incorporated by reference. In some embodiments, the modified γδ-T cells comprise the death receptor ligands or agonist antibodies or fragments thereof on the surface of the γδ-T cells. As indicated, the γδ-T cells may be genetically modified or non-genetically modified to enhance cytotoxicity. Non-genetic methods include chemically linking or attaching bispecific antibodies, linkers, engagers, etc.

Gamma/delta-T cells (γδ-T cells) are universal killer lymphocytes which recognize and kill tumor cells through their natural recognition of stress-induced determinants expressed on the surface of all cancers, regardless of their tissue of origin. As used herein, “γδ-T cells” or “gamma/delta-T cells” are any T cells that express a T cell receptor made up of one gamma chain and one delta chain.

Similar to other cytotoxic lymphocytes, γδ-T cells can kill tumor cells through: 1) classical perforin/granzyme pathways that induce necrotic cell death; or 2) can kill tumor cells by inducing apoptosis through the activation of death receptor pathways present in tumors.

Death receptor pathways can also be activated when death receptors (DRs) present on tumor cells encounter their ligands (either soluble or surface bound). Eight members of the Death Receptor family have been characterized so far and are listed below. Each death receptor responds to specific ligands. Sequences for representative death receptor genes, coding sequences, and proteins can be found on the world wide web at ncbi.nlm.nih.gov/gene.
TNFR1 (Tumor Necrosis Factor Receptor-1) also known as DR1, CD120a, p55 and p60 (a non-limiting representative gene sequence is set forth as NCBI Accession No. NG_007506.1, which is herein incorporated by reference)
Fas also known as DR2, APO-1, and CD95 (a non-limiting representative gene sequence is set forth as NCBI Accession No. NG_009089.2, which is herein incorporated by reference)
DR3 (Death Receptor-3) also known as APO-3, LARD, TRAMP, and WSL1 (a non-limiting representative gene sequence is set forth as NCBI Accession No. NG_029910.1, which is herein incorporated by reference)
TRAILR1 (TNF-Related Apoptosis-Inducing Ligand Receptor-1) also known as DR4, and APO-2 (a non-limiting representative mRNA sequence is set forth as NCBI Accession No. NM_003844.4, which is herein incorporated by reference)
TRAILR2 also known as DR5, KILLER, and TRICK2
DR6 (a non-limiting representative mRNA sequence is set forth as NCBI Accession No. AF068868.1, which is herein incorporated by reference)
EDAR (Ectodysplasin-A Receptor)
NGFR (Nerve Growth Factor Receptor)

Common ligand/receptor pairs include, but are not limited to, those provided in Table 1.

TABLE 1
Common death receptor ligand/receptor pairs

| Activating ligand | Death receptor |
|---|---|
| TNF | TNFR1/DR1/CD120a/p55 |
| FasL/CD95L | Fas/CD95/Apo1/DR2 |
| Apo3L/TWEAK | DR3/Apo3/WSL-1/TRAMP/LARD |
| TRAIL/Apo2L | TRAIL-R1/DR4 |
| | TRAIL-R2/DR5/Apo-2/TRAILCK2/KILLER |
| TRADD | DR6 |

Sequences for representative death receptor ligand genes, coding sequences, and proteins can be found on the world wide web at ncbi.nlm.nih.gov/gene. Non-limiting representative TNF genes are set forth as NCBI Gene ID No. 7124 and NCBI Accession No. NG_007462.1, each of which is incorporated by reference herein. Non-limiting representative FasL/CD95L genes are set forth as NCBI Gene ID No. 356 and NCBI Accession No. NG_007269.1, each of which is incorporated by reference herein. A non-limiting representative Apo3L/TWEAK gene and coding sequence are set forth as NCBI Gene ID No. 8742 and NCBI Accession No. AB222993.1, respectively, each of which is incorporated by reference herein. A non-limiting representative TRAIL/Apo2L gene is set forth as NCBI Gene ID No. 8743, which is incorporated by reference herein. A non-limiting representative TRADD gene is set forth as NCBI Gene ID No. 8717, which is incorporated by reference herein.

### II. Gamma Delta-T Cells Comprising Death Receptor Ligands or Antibodies or Fragments Thereof

In one embodiment, gamma/delta-T cells comprising a death receptor ligand or antibody or fragment thereof comprise a heterologous nucleic acid molecule encoding and expressing the death receptor ligand (or modified ligand) or the therapeutic agonist antibody (or fragment thereof). The ligands and/or antibodies or fragments thereof activate death receptor pathways and therefore function as agonists of at least one death receptor, potentiating the cytotoxicity of the γδ-T cells.

In another embodiment, the γδ-T cells may be modified by chemically linking or attaching antibodies or bispecific antibodies, or fragments thereof, engagers, ligands, and other receptors to recognize cancer cells and induce cell death. That is, live-cell compatible chemistry may be used to link any tumor-targeting antibody or ligand to the cells to enhance the ability of the cells to recognize and engage cancer cells. Engagers, in the art, include custom proteins that link an antibody binding domain (e.g., CD19 or CD20) to a construct that includes a protein that specifically binds to the γδ-T cell receptor. See, for example, WO2019156566, herein incorporated by reference. In one embodiment, an engager molecule (Anti-TRGV9/anti-CD123), a bispecific antibody that can simultaneously bind to the Vγ9 chain of the Vγ9Vδ2+γδT cell receptor and to AML target antigen, CD123, to selectively recruit Vγ9+γδT cells (rather than pan T cells) to target AML blasts can be used.

In contrast to conventional methods of linking the targeting moieties directly to the γδ-T cell receptor, the methods of the invention link the targeting moieties to non-functioning surface molecules of the T cells, including but not limited to HLA class I or class II, CD52, and the like. In fact, in some embodiments, the modified cells do not link moieties to the T cell receptor (TCR) itself, therefore avoiding unwanted cross-linking of the TCR on the γδ-T cell resulting in strong activation signals and killing of the γδ-T cells via activation of induced cell death. In these embodiments, activation-induced cell death of the γδ-T cells is avoided.

The death receptor ligand whether expressed or attached can work in conjunction with 1) targeting modifications or 2) other death receptor ligands (either expressed or attached). In this manner, for example, a gamma/delta T cell modified to target CD19 (either by a CAR or by a bispecific antibody) may also be modified to contain a death receptor ligand or death receptor engager. As used herein, the term “nucleic acid molecule” encompasses single-stranded and double-stranded molecules or molecules comprising single-stranded and double-stranded regions. The term also encompasses RNA, DNA or combinations thereof and the nucleic acid molecule may comprise modified nucleotides or non-naturally occurring nucleotide analogs.

As used herein, “heterologous” in reference to a sequence is a sequence that originates from a foreign species, or, if from the same species, is substantially modified from its native form in composition and/or genomic locus by deliberate human intervention.

An “engineered gamma/delta-T cell” is a gamma/delta-T cell that comprises a heterologous nucleic acid molecule.

Death receptor ligands including, but not limited to the ligands listed herein, or variants of naturally occurring ligands may be expressed by or attached to γδ-T cells. These ligands that are now on the surface of the γδ-T cells, will engage the death receptor present on tumor cells which will potentiate the killing of targets through the induction of apoptosis.

In some embodiments, the modified γδ-T cells express or are attached to agonist antibodies or fragments thereof that activate death receptors. Several agonist antibodies have been specifically developed as they can induce signaling through death receptors. For example, Table 1 of Wiezorek et al. (2010) Clinical Cancer Research, DOI: 10.1158/1078-0432.CCR-09-1692 (which is herein incorporated by reference), lists several antibodies and a recombinant human soluble protein called dulanermin (corresponding to amino acids 114-281 of the Apo2 ligand/tumor necrosis factor-related apoptosis-inducing ligand (RhApo2L/TRAIL) with potential antineoplastic activity).

In certain embodiments, antibody fragments or domains expressed by or on the modified γδ-T cells can provide the same agonist signal as the whole antibody. These antibody fragments or domains that are chemically attached or expressed constitutively on the γδ-T cells, will engage the death receptor present on tumor cells which will potentiate the killing of targets through induction of apoptosis. In some embodiments, Fv/CDRs may be used to function as an agonist of the death receptors and induce apoptosis. FcR-mediated cross linking may be engineered into the chimeric receptor so that binding domains are built in such a way that they are spatially approximated (through the use of linkers) such that they can provide cross-linking activity. As noted above, the death receptor ligand of the modified γδ-T cells can work in conjunction with targeting modifications or other death receptor ligands that are introduced by genetic or non-genetic means.

The term “antibody” is used in the broadest sense and covers fully assembled antibodies, antibody fragments that can bind antigen (e.g., Fab, F(ab′) 2, Fv, single chain antibodies, diabodies, antibody chimeras, hybrid antibodies, bispecific antibodies, humanized antibodies, and the like), and recombinant peptides comprising the forgoing.

“Antibody fragments” comprise a portion of an intact antibody, preferably the antigen-binding or variable region of the intact antibody. Examples of antibody fragments include Fab, F(ab′) 2, and Fv fragments; diabodies; linear antibodies (Zapata et al. (1995) Protein Eng. 10:1057-1062); single-chain antibody molecules; and multispecific antibodies formed from antibody fragments.

In one embodiment, the antibody is a monoclonal antibody. By “monoclonal antibody” is intended an antibody obtained from a population of substantially homogeneous antibodies, that is, the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, such as those produced by a clonal population of B-cells, and is not to be construed as requiring production of the antibody by any particular method.

By “epitope” is intended the part of an antigenic molecule to which an antibody is produced and to which the antibody will bind. Epitopes can comprise linear amino acid residues (i.e., residues within the epitope are arranged sequentially one after another in a linear fashion), nonlinear amino acid residues (referred to herein as “nonlinear epitopes”—these epitopes are not arranged sequentially), or both linear and nonlinear amino acid residues.

The modified gamma/delta-T cells can comprise soluble death receptor ligands and/or agonist antibodies or fragments thereof or the modified cells can express the ligands and/or agonist antibodies or fragments thereof on their surface. Any method known in the art to tether a protein to the surface of the γδ-T cell can be used, including but not limited to, fusion of a transmembrane domain to the ligand and/or agonist antibody or fragment thereof. In some embodiments, the engineered gamma/delta-T cells are CAR-T cells in that they express a chimeric antigen receptor (CAR) with agonist activity against a death receptor. In other embodiments, the CAR_T cells may comprise moieties (bispecific antibodies, linkers, etc.) that can be used to engage death receptors present on tumor cells.

### III. Pharmaceutical Compositions

Pharmaceutical compositions comprising the modified gamma/delta-T cells comprising a death receptor ligand and/or agonist antibody or fragment thereof and a pharmaceutically acceptable carrier are provided.

As used herein “pharmaceutically acceptable carriers” are well known to those skilled in the art and include, but are not limited to, 0.01-0.1 M, or 0.05M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

Modified γδ-T cells can be formulated for administration through a variety of means, including but not limited to, intravenous (IV) infusion, intra-arterial infusion, intra-peritoneal infusion, intra-pleural infusion, direct intratumoral injection, intracranial injection, introduction into the cerebrospinal fluid (CSF) by lumbar puncture, and intraocular injection.

### IV. Methods of Engineering Gamma Delta-T Cells to Express Death Receptor Ligands and or Antibodies or Fragments Thereof

Any method known in the art to engineer a cell, such as a gamma/delta-T cell, to express a recombinant protein, such as a death receptor ligand and/or agonist antibody or fragment thereof, can be used in the presently disclosed compositions and methods. Any method can be used to introduce a nucleic acid encoding a death receptor ligand and/or agonist antibody or fragment thereof into a γδ-T cell such that the nucleic acid enters the cell and is expressed by the cell. The γδ-T cell can express the death receptor ligand and/or agonist antibody or fragment thereof transiently or stably (wherein the coding sequence is incorporated into the genome of the γδ-T cell). Generally, stable expression is utilized.

Molecular techniques, including gene editing technologies, are known in the art. In some embodiments a heterologous nucleic acid molecule encoding the death receptor ligand and/or agonist antibody or fragment thereof that is introduced into a γδ-T cell further comprises at least one regulatory element (e.g., promoter, enhancer). In one embodiment, the DNA encoding the death receptor ligand and/or agonist antibody or fragment thereof can be inserted into a vector, and the vector can be introduced into a cell. For example, a virus vector such as a retrovirus vector (including an oncoretrovirus vector, a lentivirus vector, and a pseudo type vector), an adenovirus vector, an adeno-associated virus (AAV) vector, a simian virus vector, a vaccinia virus vector or a sendai virus vector, an Epstein-Barr virus (EBV) vector, and a HSV vector can be used. A virus vector lacking replicating ability so as not to self-replicate in an infected cell is preferably used. A non-virus vector can also be used in the present invention in combination with a liposome and a condensing agent such as a cationic lipid as described in WO 96/10038, WO 97/18185, WO 97/25329, WO 97/30170 and WO 97/31934 (which are incorporated herein by reference). The nucleic acid encoding a death receptor ligand and/or agonist antibody or fragment thereof can also be introduced into a cell by calcium phosphate transduction, DEAE-dextran, electroporation, or particle bombardment.

In some embodiments, the nucleic acid encoding a death receptor ligand and/or agonist antibody or fragment thereof can be introduced into a γδ-T cell and incorporated into its genome via gene editing, including but not limited to gene editing techniques involving programmable nucleases, such as zinc-finger nucleases, transcription activator-like effector nucleases (TALENs), CRISPR-Cas nucleases, and engineered homing endonucleases such as those described in U.S. Pat. Nos. 8,679,359; 8,771,945; 8,795,965; 8,865,406; 8,871,445; 8,889,356; 8,895,308; 8,906,616; 8,932,814; 8,945,839; 8,993,233; 8,999,641; 9,840,713; U.S. application Ser. Nos. 15/947,680; 15/947,700; 15/947,718; 15/981,807; 15/981,808; 15/981,809; 16/136,159; 16/136,165; 16/136,168; 16/136,175; 16/276,361; 16/276,365; 16/276,368; 16/276,374; (all of which are herein incorporated by reference) and the like. See also, Killing Mechanisms of Chimeric Antigen Receptor (CAR) T Cells Mohamed-Reda Benmebarek et al. Int. J. Mol. Sci. 2019, 20, 1283; doi: 10.3390/ijms20061283, which is herein incorporated by reference.

The engineered γδ-T cells can be expanded as described in International Patent Application Publication No. WO 2020/065584, herein incorporated by reference. γδ-T cells are extremely sensitive to activation-induced cell death (AICD) and will undergo apoptosis when overstimulated or overactivated (Lopez et al., 2000, Blood 96:3827-3837; Guo et al., 2002 Cancer Immunology and Immunotherapy 50:625-637). This poses a particular challenge when attempting to genetically modify γδ-T cells as such cells (and T cells in general) must be induced to proliferate in order to be efficiently edited, and also must be induced to expand following genetic modification. Conventional methods commonly used to activate γδ-T cells prior to genetic modification (or subsequent activation used to induce proliferation of these cells) quite often lead to γδ-T cell death. In contrast, activation and expansion protocols employing direct-acting phosphoantigens (including, but not limited to C-HDMAPP) are optimized to stimulate—but not over-stimulate—Vγ9δ2 γδ-T cells. This is possible as direct-acting phosphoantigens can be precisely titrated at nanomolar concentrations to promote optimal γδ-T cell activation while avoiding overstimulation. Such concentrations include, but are not limited to, about 0.1 nM, about 1 nM, about 5 nM, about nM, about 20 nM, about 50 nM, about 75 nM, or about 100 nM. Thus, these activation methods predictably and routinely can activate (without overactivating and killing) γδ-T cells prior to their gene editing, and following editing, can be used to expand these γδ-T cells which results in high yields of viable genetically modified γδ-T cells.

In some embodiments, cell cultures are initiated by seeding PBMC into tissue culture flasks to which recombinant human IL-2 is added. γδ-T cell expansion is initiated by the addition of C-HDMPP at optimized concentrations. Fresh media is added as needed. Human IL-2 is added every 5 days. Cultures are maintained for at least about 18 to at least about 21 days, or greater. Therefore, the use of direct-acting phosphoantigen compound allows for optimal stimulation (but not overstimulation) of γδ-T cells and optimized introduction of genes by various methods (including but not limited to viral transduction; electroporation) leading to the generation of larger numbers of more viable cells with superior gene expression.

The ability to carefully control the activation/stimulation of our γδ-T cells using carefully titrated phosphoantigens allows precise regulation or moderation of the expansion of cells over time. This allows the editing of cells (introduction of genes) at optimum time in relation to stimulation. Under conditions that can be carefully controlled, it has been determined that if gene introduction is performed immediately prior to culture stimulation (day 0), gene expression does not occur due to massive cellular death. Similarly, if gene introduction is performed 24 hours after culture stimulation (day 1), poor gene expression occurs. However, if gene introduction is performed at later times (at least 2 days, at least 3 days, at least 4 days, at least 5 days, at least 6 days, or greater than 6 days after culture stimulation), gene introduction occurs with corresponding cell expansion of modified cells. In some embodiments of the presently disclosed methods, gene introduction occurs about 2 days, about 3 days, about 4 days, about 5 days, about 6 days, about 7 days, or more after stimulation of the γδ-T cells.

The engineered γδ-T cells may be administered in combination with ligands or antibody fragments or domains. See, e.g., WO 2020/065584.

In some embodiments, the death receptors that may be present on γδ-T cells can be deleted or inactivated so as to prevent undesirable activation-induced cell death during culture of the cells.

### V. Methods of Treating Cancer

Provided herein are methods of treating cancer in a subject. Such methods comprise administering to a subject in need thereof a therapeutically effective amount of modified gamma/delta-T cells expressing a death receptor ligand and/or agonist antibody or fragment thereof.

The modified cells of the invention are useful in the treatment of cancer. The term “cancer” is intended to be broadly interpreted and it encompasses all aspects of abnormal cell growth and/or cell division. Examples include: carcinoma, including but not limited to adenocarcinoma, squamous cell carcinoma, adenosquamous carcinoma, anaplastic carcinoma, large cell carcinoma, small cell carcinoma, and cancer of the skin, breast, prostate, bladder, vagina, cervix, uterus, liver, kidney, pancreas, spleen, lung, trachea, bronchi, colon, small intestine, stomach, esophagus, gall bladder; sarcoma, including but not limited to chondrosarcoma, Ewing's sarcoma, malignant hemangioendothelioma, malignant schwannoma, osteosarcoma, soft tissue sarcoma, and cancers of bone, cartilage, fat, muscle, vascular, and hematopoietic tissues; lymphoma and leukemia, including but not limited to mature B cell neoplasms, such as chronic lymphocytic leukemia/small lymphocytic lymphoma, B-cell prolymphocytic leukemia, lymphomas, and plasma cell neoplasms, mature T cell and natural killer (NK) cell neoplasms, such as T cell prolymphocytic leukemia, T cell large granular lymphocytic leukemia, aggressive NK cell leukemia, and adult T cell leukemia/lymphoma, Hodgkin lymphomas, and immunodeficiency-associated lymphoproliferative disorders; germ cell tumors, including but not limited to testicular and ovarian cancer; blastoma, including but not limited to hepatoblastoma, medulloblastoma, nephroblastoma, neuroblastoma, pancreatoblastoma, pleuropulmonary blastoma and retinoblastoma. The term also encompasses benign tumors.

As used herein, the terms “treat”, “treating”, and “treatment” have their ordinary and customary meanings, and include one or more of: blocking, ameliorating, or decreasing in severity and/or frequency a symptom of cancer in a subject, and/or inhibiting the growth, division, spread, or proliferation of cancer cells, or progression of cancer (e.g., emergence of new tumors) in a subject. Treatment means blocking, ameliorating, decreasing, or inhibiting by about 1% to about 100% versus a subject in which the methods of the present invention have not been practiced. Preferably, the blocking, ameliorating, decreasing, or inhibiting is about 100%, 99%, 98%, 97%, 96%, 95%, 90%, 80%, 70%, 60%, 50%, 40%, 30%, 20%, 10%, 5% or 1% versus a subject in which the methods of the present invention have not been practiced.

As used herein, “subject” is intended any animal (i.e. mammals) such as, humans, primates, rodents, agricultural and domesticated animals such as, but not limited to, dogs, cats, cattle, horses, pigs, sheep, and the like, in which one desires to treat cancer. In one embodiment, the subject is a mammal. In a specific embodiment, the subject is a human.

A therapeutically effective amount of engineered or non-genetically modified γδ-T cells is provided for use in the methods provided herein. A “therapeutically effective amount,” “therapeutically effective dose,” or “effective amount” as used herein refers to that amount which provides a therapeutic effect for a given condition and administration regimen. Thus, the phrase “therapeutically effective amount” is used herein to mean an amount sufficient to cause an improvement in a clinically significant condition in the host. In particular aspects, a “therapeutically effective amount” refers to an amount of modified γδ-T cells that, when administered, brings about a positive therapeutic response with respect to treatment of a subject for a cancer. A positive therapeutic response in regard to treating a cancer includes curing or ameliorating the symptoms of the disease. In the present context, a deficit in the response of the subject can be evidenced by continuing or spreading of the cancer. An improvement in a clinically significant condition in the subject includes a decrease in the size of a tumor, increased necrosis of a tumor, clearance of the tumor from the host tissue, reduction or amelioration of metastasis, or a reduction in any symptom associated with the cancer.

An “antitumor response” refers to a positive therapeutic response in regard to treating a cancer and includes curing or ameliorating the symptoms of the disease. An antitumor response in the subject includes a decrease in the size of a tumor, increased necrosis of a tumor, clearance of the tumor from the host tissue, the presence of anti-tumor immune cells in the subject, the presence of immune cells in or adjacent to the tumor, reduction or amelioration of metastasis, or a reduction in any symptom associated with the cancer.

In the therapeutic methods and compositions provided herein, a therapeutically effective dosage of the active component is provided. A therapeutically effective dosage can be determined by the ordinary skilled medical worker based on patient characteristics (age, weight, sex, condition, complications, other diseases, etc.), as is well known in the art. Furthermore, as further routine studies are conducted, more specific information will emerge regarding appropriate dosage levels for treatment of various conditions in various patients, and the ordinary skilled worker, considering the therapeutic context, age and general health of the recipient, is able to ascertain proper dosing. Generally, for intravenous injection or infusion, dosage may be lower than for intraperitoneal, intramuscular, or other route of administration. The dosing schedule may vary, depending on the circulation half-life, and the formulation used. The compositions are administered in a manner compatible with the dosage formulation in the therapeutically effective amount. Precise amounts of active ingredient required to be administered depend on the judgment of the practitioner and are peculiar to each individual. However, suitable dosages may range from about 0.1×10⁶ cells/kg (of recipient weight) to about 100×10⁶ cells/kg, preferably about 0.5×10⁶ cells/kg to about 10×10⁶ cells/kg, and more preferably one to several 10⁶ cells/kg per day and depend on the route of administration. Suitable regimes for initial administration and booster shots are also variable but are typified by an initial administration followed by repeated doses at one or more hour intervals by a subsequent injection or other administration. Alternatively, continuous intravenous infusions are contemplated.

Generally, compositions of γδ-T cells are prepared in the form suited for the route of administration thereof, for example in the form of injections, transfusions or like liquids or solutions. The liquid or solution forms, inclusive of injections, can be prepared in the same manner as in preparing various conventional pharmaceutical preparations or cellular therapies as described above. The carrier to be used may be any of various pharmaceutically acceptable carriers (diluents) well known in the art. A non-limiting examples thereof is PBS. In preparing the above-mentioned liquid or solution forms, various technologies currently in general use in preparing various transfusions can be used. The γδ-T cell compositions may be prepared just prior to use. The γδ-T cell compositions are administered at respective predetermined doses via a predetermined route(s) of administration according to the method described herein. For example, the number of cells for infusion into a subject can be administered in the range from about 1×10³ to about 1×10¹⁰, from about 1×10⁴ to about 1×10¹⁰, from about 1×10⁵ to about 1×10¹⁰, form about 1×10⁶ to about 1×10¹⁰, form about 1×10⁷ to about 1×10¹⁰, form about 1×10⁸ to about 1×10¹⁰, from about 1×10³ to about 1×10⁹, form about 1×10⁵ to about 1×10⁹, form about 1×10⁶ to about 1×10⁹, form about 1×10⁴ to about 1×10⁸, form about 1×10⁶ to about 1×10⁸, or form about 1×10⁵ to about 1×10⁷ per infusion. The methods of treating cancer provided herein can encompass administration of treatment via any parenteral route, including, but not limited, to intramuscular, intraperitoneal, intravenous, and the like.

It is recognized that the method of treatment may comprise a single administration of a therapeutically effective dose of the therapy or multiple administrations of a therapeutically effective dose of the therapy. Moreover, the treatment can be accomplished with varying doses as well as dosage regimens and using cells from different donors (as described below).

In some embodiments, the γδ-T cells that are modified to include a death receptor ligand and/or agonist antibody or fragment thereof are derived from the subject that are then administered the modified γδ-T cells, thus comprising autologous cells. In other embodiments, the subject is administered allogeneic modified γδ-T cells wherein the donor for the γδ-T cells that are modified (engineered or chemical attachments) is not the subject that is administered the modified cells, but the donor is of the same species as the subject. In some of these embodiments, the donor of the γδ-T cells is a full human leukocyte antigen (HLA) mismatch as compared to the subject receiving the modified γδ-T cells. By full HLA mismatch is meant that the subject and the donor do not share any HLA antigens. In other embodiments, the donor is a partial HLA mismatch. By partial HLA mismatch is meant that at least one HLA antigen is different between the subject and the donor.

In some embodiments, the subject is pretreated with an amino-bisphosphonate prior to administration of the modified γδ-T cells. Amino-bisphosphonates (such as zoledronic acid) can act indirectly on γδ-T cells by causing bystander cells to release isopentenyl pyrophosphate (IPP). This happens since in bystander cells, zoledronic acid inhibits the enzyme farnesyl pyrophosphate synthase (FPPS), a critical enzyme in the mevalonate biosynthetic pathway. Disruption of the pathway results in the accumulation of IPP which is eventually released from cells which then in turn, stimulates γδ-T cells. Tumor cells exposed to amino-bisphosphonates (such as zoledronic acid) also can be made to release IPP and related phosphoantigens. As tumor cells already preferentially produce IPP as a result of their dysregulated state, then the effect of an amino-bisphosphonate is even greater. It is this enhanced release of IPP by tumor cells first exposed to an amino-bisphosphonate that makes these cells more sensitive to killing by γδ-T cells. In addition, this IPP can serve as a chemoattractant to γδ-T cells, making them even more potent in vivo. In one embodiment, the method of treating cancer further comprises administering an amino-bisphosphonate to the subject prior to the administration of the modified γδ-T cells. In a specific embodiment, the amino-bisphosphonate is zoledronic acid.

In some embodiments, the subject is administered a lymphodepletion treatment prior to the administration of the modified γδ-T cells. A “lymphodepletion treatment” as used herein, is any treatment that results in lymphodepletion in a subject. Lymphodepletion treatments include, but are not limited to, for example, methylprednisolone, radiation, low dose total body irradiation, etoposide, cisplatin, doxorubicin, 5-Fluorouracil, vincristine, bortezomib, oxaliplatin, or any lymphodepleting chemotherapeutic agents, including cyclophosphamide, fludarabine or melphalan. In one embodiment, the lymphodepletion treatment comprises administering one or more chemotherapeutic agents. In specific embodiments, the chemotherapeutic agents comprise cyclophosphamide, fludarabine or melphalan. In another embodiment, the lymphodepletion treatment comprises low dose total body irradiation.

The methods provided herein may comprise a combination therapy of modified γδ-T cells and one or more therapeutic agents. The term “combination” or “combination therapy” is used herein in its broadest sense and means that a subject is treated with at least two therapeutic regimens. The timing of administration of the different therapeutic regimens can be varied so long as the beneficial effects of the combination of these therapeutic regimens are achieved. Treatment with modified γδ-T cells in combination with one or more therapeutic agents can be simultaneous (concurrent), consecutive (sequential), or a combination thereof. Therefore, a subject undergoing combination therapy can receive both modified γδ-T cells and one or more therapeutic agents at the same time (i.e., simultaneously), or at different times (i.e. sequentially, in either order, on the same day, or on different days), as long as the therapeutic effect of the combination of both is caused in the subject undergoing therapy. Where the modified γδ-T cells or one or more therapeutic agents are administered simultaneously, they can be administered as separate pharmaceutical compositions, each comprising either modified γδ-T cells or therapeutic agent, or can be administered as a single pharmaceutical composition comprising both of these agents. In some embodiments, one or more therapeutic agents are administered as a pre-treatment prior to administration of the modified γδ-T cells. The one or more therapeutic agents may be administered to the subject at least 2 hours, 4 hours, 6 hours, 8 hours, 12 hours, 24 hours, 48 hours, 72 hours, or more prior to the administration of the modified γδ-T cells.

The combination therapy provided herein can also be achieved intermittently. By “intermittent combination therapy” is intended a period of combination therapy with modified γδ-T cells and one or more therapeutic agents, followed by a time period of discontinuance, which is then followed by another period of combination therapy with modified γδ-T cells and one or more therapeutic agents, and so forth.

In the methods provided herein, the modified γδ-T cells may be combined with one or more therapeutic agents to potentiate the in vivo killing activity and/or homing ability of the γδ-T cells. Exposing tumor cells to certain chemicals, including but not limited to bisphosphonates, chemotherapeutic agents, etc. can cause tumor cells to create chemical gradients that attract γδ-T cells. These chemoattractant gradients can induce the homing of unmodified or modified γδ-T cells to tumor sites (Benzaïd et al., 2011 Cancer Res 71 (13): 4562-4572). As such, tumors, including tumors that are relatively resistant to treatment, can be sensitized to killing by γδ-T cells. γδ-T cell sensitizing agents include, but are not limited to, chemotherapeutic agents, including etoposide, cisplatin, doxorubicin, 5-Fluorouracil, vincristine, bortezomib and oxaliplatin; cyclophosphamide; fludarabine; hypomethylating agents, including 5-azacitadine; small molecules, including ibrutinib, venetoclax; therapeutic antibodies, including rituximab, trastuzumab, nivolumab, pembrolizumab, and ipilimumab; or amino-bisphosphonates, including zoledronic acid. In one embodiment, the methods provided herein further comprise administering to the subject one or more of etoposide, cisplatin, doxorubicin, 5-Fluorouracil, vincristine, bortezomib, oxaliplatin or ibrutinib.

In one embodiment, compounds that block anti-apoptosis pathways thereby promoting tumor cell death (including, but not limited to venetoclax) can be combined with the modified γδ-T cells of the invention potentiating the ability to kill tumor or cancer cells through the enhanced induction of apoptosis via death receptor pathways. See, for example, Murakami et. al., (2020) Hematological Oncology 38 (5): 705-714, doi.org/10.1002/hon.2794), herein incorporated by reference.

The modified γδ-T cells may also be administered as a combination therapy with other cellular therapies in order to potentiate anti-tumor activity. The engineered or non-genetically modified γδ-T cells may be administered concurrently or sequentially with any number of other cell types, including, but not limited to, γδ-T cells of the Vδ1 variety, or any γδ-T cell subset that is non-Vδ2; NK cells; or CAR-T cells (either αβ-T cell or NK cell or γδ-T cell derived, or any other CAR-modified cell). In one embodiment, the methods provided herein further comprise administering one or more additional cellular therapies. In some methods, the one or more additional cellular therapies are from the same donor as the γδ-T cells.

One may administer the modified γδ-T cells in conjunction with one or more pharmaceutical compositions used for treating cancer, including but not limited to chemotherapeutic agents. This can include the use of checkpoint inhibitor antibodies, including but not limited to pembrolizumab and ipilimumab, designed to augment immune function of effector lymphocytes, including adoptively transferred modified γδ-T cells (Grosser et al., 2019 Cancer Cell 36 (5): P471-482). In some embodiments, the modified γδ-T cells can be administered in conjunction with antibodies that selectively activate γδ-T cells including, but not limited to a humanized, anti-BTN3A (also known as CD277) monoclonal antibody that selectively activates γ9δ2 T cells (Gassert et al., 2021 Science Translational Medicine 13 (616), DOI: 10.1126/scitranslmed.abj0835). Administration may be simultaneous (for example, administration of a mixture of the modified γδ-T cells and a chemotherapeutic agent), or may be in seriatim.

Those skilled in the art recognize that the methods of therapy disclosed herein may be used before, after, or concurrently with other forms of oncotherapy. Such oncotherapy can include chemotherapy regimens or radiation treatment.

That which is claimed:

1. A gamma/delta-T (γδ-T) cell comprising a heterologous death receptor ligand and/or death receptor agonist antibody or fragment thereof.

2. The γδ-T cell of claim 1, wherein said heterologous death receptor ligand and/or agonist antibody or fragment thereof comprises a bispecific antibody, an engager, or a linker.

3. The γδ-T cell of claim 1 or 2, wherein said heterologous death receptor ligand and/or agonist antibody or fragment thereof is not linked to the gamma/delta-T cell receptor.

4. The γδ-T cell of claim 1, wherein said γδ-T cell comprises a heterologous nucleic acid encoding said death receptor ligand and/or death receptor agonist antibody or fragment thereof.

5. The γδ-T cell of any one of claims 1-4, wherein said γδ-T cell is a chimeric antigen receptor (CAR)-T cell.

6. A pharmaceutical composition comprising a pharmaceutically acceptable carrier and a γδ-T cell of any one of claims 1-5.

7. A method of engineering a gamma/delta-T (γδ-T) cell to express a heterologous death receptor ligand and/or a death receptor agonist antibody or fragment thereof, wherein said method comprises introducing into a γδ-T cell a heterologous nucleic acid molecule encoding a death receptor ligand and/or a death receptor agonist antibody or fragment thereof, thereby producing an engineered γδ-T cell.

8. The method of claim 7, wherein said heterologous nucleic acid molecule is in a vector.

9. The method of claim 8, wherein said vector is a virus vector.

10. The method of any one of claims 7-9, wherein said heterologous nucleic acid molecule is introduced into said γδ-T cell and incorporated into its genome via gene editing.

11. The method of any one of claims 7-10, wherein said method further comprises stimulating said γδ-T cell prior to introducing said heterologous nucleic acid molecule.

12. The method of claim 11, wherein said stimulating occurs at least 1 day prior to introducing said heterologous nucleic acid molecule.

13. The method of claim 12, wherein said stimulating occurs at least 2 days prior to introducing said heterologous nucleic acid molecule.

14. The method of any one of claims 7-13, wherein said method further comprises stimulating said γδ-T cell.

15. A method of treating a cancer in a subject in need thereof, said method comprising administering a therapeutically effective amount of a γδ-T cell of any one of claims 1-5 or the pharmaceutical composition of claim 6.

16. The method of claim 15, wherein said subject is a human.

17. The method of claim 15 or 16, wherein said γδ-T cell is derived from said subject.

18. The method of claim 15 or 16, wherein said γδ-T cell is derived from a donor that is not said subject, and wherein said donor is the same species as said subject.

19. The method of embodiment 18, wherein said donor is a partial or a full human leukocyte antigen (HLA) mismatch compared to said subject.

20. The method of any one of embodiments 15-19, wherein said method further comprises treating said subject with an amino-bisphosphonate prior to administration of said γδ-T cell or said pharmaceutical composition.

21. The method of any one of embodiments 15-19, wherein said method further comprises administering a lymphodepletion treatment to said subject prior to administration of said γδ-T cell or said pharmaceutical composition.
